(19) United States
(12) Patent Application Publication
(10) Pub. No.: US 2026/0360551 A1
(43) Pub. Date: Nov. 12, 2026

(54) CARDIOVASCULAR RISK DETECTION USING KOROTKOFF SOUNDS

(71) Applicant: Hong Kong Applied Science and Technology Research Institute Co., Ltd., Shatin (HK)

(72) Inventors: Chun Ming Chan, Kowloon (HK); Chun Yiu Yuen, Tseung Kwan O (HK)

(21) Appl. No.: 19/200,400

(22) Filed: May 6, 2025

(51) Int. Cl. G16H 50/30; G16H 10/60

(52) U.S. Cl. CPC G16H 50/30; G16H 10/60

(57) ABSTRACT

A method of operation of a multi-sequence former for enhanced sequence processing and classification includes generating a low-level descriptor (LLD) features associated with Korotkoff sounds (K-sounds) indicated by patient measurement data. The method further includes performing, by an input handling stage of the multi-sequence former, embedding of at least some of the LLD features into a vector space to generate a plurality of LLD feature vectors. The method further includes determining, by a multi-head sequence attention module of the multi-sequence former, sequence attention scores and temporal attention scores associated with the plurality of LLD feature vectors. The method further includes generating, by the multi-sequence former, an output based on the sequence attention scores and the temporal attention scores. The output indicates a patient cardiovascular risk assessment associated with the K-sounds.

### TECHNICAL FIELD

The disclosure relates generally to cardiovascular risk detection and, more particularly, to cardiovascular risk detection using Korotkoff sounds.

### BACKGROUND

Cardiovascular disease is a leading cause of preventable death worldwide. Risk factors for cardiovascular disease include lifestyle factors, such as poor diet, physical inactivity, smoking, alcohol use, stress, and poor sleep quality. Further, some individuals may be genetically susceptible to cardiovascular disease even if such lifestyle factors are controlled. For example, an individual may be genetically prone to high blood serum cholesterol via familial hypercholesterolemia, which may increase risk of cardiovascular disease.

Treatment of cardiovascular disease is typically resource-intensive. For example, treatment of a myocardial infarction (also known as a heart attack) may involve hospitalization and treatment by highly-trained medical professionals to perform complex procedures, such as angioplasty. Further, the prevalence of cardiovascular disease may introduce a large amount of strain on healthcare resources and may reduce availability of such healthcare resources for other patients.

To reduce or prevent cardiovascular disease, patients may be screened for the presence of various risk factors. Risk screening modalities include blood pressure measurements, cholesterol and lipid testing, and calcium scoring using imaging techniques. Such risk assessment modalities may nonetheless fail to detect or indicate symptoms of cardiovascular disease until relatively advanced stages of cardiovascular disease. Further, some risk assessment modalities may be relatively expensive and inaccessible to patients. For example, calcium scoring may be relatively costly and may not be widely used in some countries. As a result, cardiovascular disease remains a leading cause of mortality worldwide.

### SUMMARY

Aspects of the disclosure are related to cardiovascular risk detection based on Korotkoff sounds (K-sounds) that are created by the circulatory system of a patient as a result of changes in pressure against the walls of blood vessels. Such K-sounds may be recorded during examination of the patient, such as using an auscultation device (e.g., a stethoscope) or a sphygmomanometer (e.g., an arm-worn blood pressure gauge). A cardiovascular risk assessment system may receive data associated with a patient and may analyze the data to determine low-level descriptor (LLD) features associated with K-sounds indicated by the data. The cardiovascular risk assessment system may analyze the LLD features to determine risk of cardiovascular disease associated with the patient. Analyzing the LLD features may include determining sequence attention scores for features related to the same timepoint and determining temporal attention scores for features across a time interval.

Using both sequence attention and temporal attention may enhance the accuracy and efficiency of assessment of cardiovascular risk. To illustrate, by analyzing such different sequences of the LLD features, the cardiovascular risk assessment system may detect relationships between different LLD features, such as inter-sequence dependencies. Such dependencies may improve accuracy of detecting or classifying risk of cardiovascular disease. Further, by analyzing the progression of features over time, the cardiovascular risk assessment system may also detect changes and trends associated with different time points that may indicate cardiovascular risk.

In some examples, a convolutional neural network (CNN)-free one-dimensional (1D) transformer may determine the sequence attention scores and the temporal attention scores. Use of a CNN-free 1D transformer may facilitate efficient detection of local patterns and relatively short-term dependencies among features. Further, the CNN-free 1D transformer may have a multi-head configuration in which one or more sequence attention heads apply sequence attention to the LLD features and in which one or more temporal attention heads apply temporal attention to the LLD features. In some examples, the multiple sequence attention heads may attend to interactions between sequences of different LLD features, leading to more accurate and nuanced representation of the patient data. In some examples, the multiple temporal attention heads may attend to different temporal patterns, which may facilitate a fine-grained analysis of time series data. Such a fine-grained analysis may increase temporal sensitivity, which may be advantageous in detecting subtle variations in K-sounds that may be associated with risk of cardiovascular disease, such as heart failure (HF).

One or more features associated with such a cardiovascular risk assessment system may achieve one or more of the following benefits. In some examples, such a cardiovascular risk assessment system may enable screening of patients for cardiovascular risk using relatively low-cost modalities. For example, because many examinations of patients may already routinely involve use of a stethoscope and blood pressure gauge, K-sounds can be quickly and inexpensively detected using existing modalities, which may reduce or avoid more expensive modalities, such as calcium scoring. Further, analysis of the K-sounds may be performed using a relatively inexpensive computing system, such as using a CNN-free 1D transformer that determines sequence attention scores and temporal attention scores based on patient data. Accordingly, cardiovascular risk may be assessed more accurately using relatively low-cost and low-complexity techniques.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows a system for cardiovascular risk detection using Korotkoff sounds (K-sounds) in accordance with some aspects of the disclosure.

FIG. 2 shows example pre-processing operations in accordance with some aspects of the disclosure.

FIG. 3 shows an example of a genetic selection procedure in accordance with some aspects of the disclosure.

FIG. 4 shows an example of a masking operation in accordance with some aspects of the disclosure.

FIG. 5 shows examples of features that may be associated with an artificial intelligence (AI) engine in accordance with some aspects of the disclosure.

FIG. 6 shows an example of an embedding process in accordance with some aspects of the disclosure.

FIG. 7 shows examples of operations in accordance with some aspects of the disclosure.

FIG. 8 shows an example of a method in accordance with some aspects of the disclosure.

In the figures, like reference numbers refer to like elements. It should be understood that the figures may be presented partially or diagrammatically. Some figures are presented by omitting features in order that components or operations may not be obscured. While the figures present several example components, not every component may be necessary in every embodiment. Nor does every embodiment need to be arranged exactly as in the figures in order to practice the principles disclosed herein. Alternative constructions are described herein for purposes of example only, and should not be construed as limiting the scope of this disclosure or the claims.

### DETAILED DESCRIPTION OF THE INVENTION

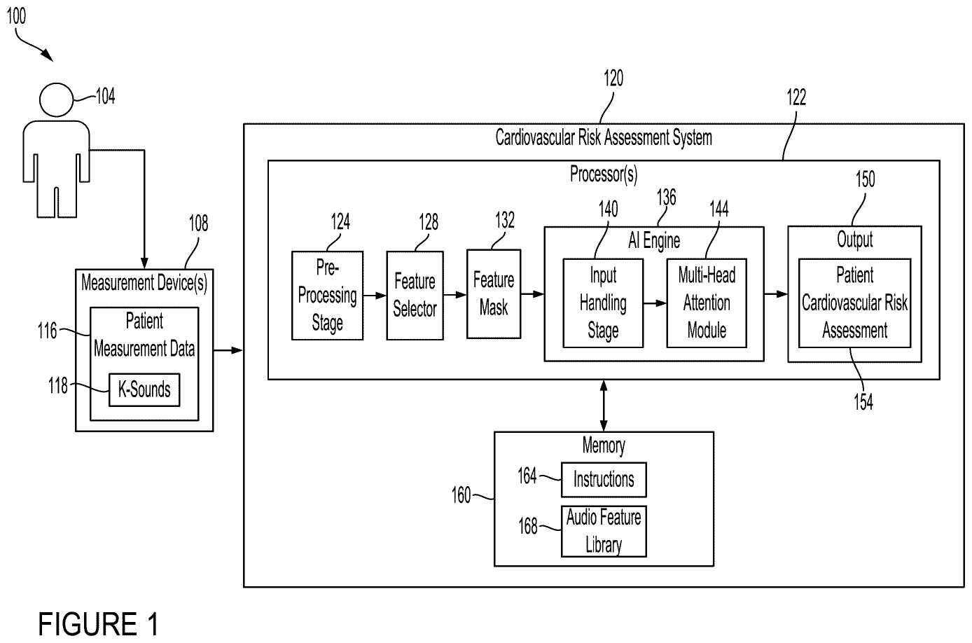

FIG. 1 shows a system 100 for cardiovascular risk detection using Korotkoff sounds (K-sounds) in accordance with some aspects of the disclosure. The system 100 may include one or more measurement devices 108 and a cardiovascular risk assessment system 120.

In some implementations, the one or more measurement devices 108 may include an auscultation device, such as a stethoscope. Alternatively, or in addition, the one or more measurement devices 108 may include a sphygmomanometer, such as an arm-worn blood pressure gauge. Further, the one or more measurement devices 108 may include an audio recording device, such as a microphone. For example, an output of the auscultation device or sphygmomanometer may be connected to an input of the audio recording device such that the audio recording device is able to record audio detected by the auscultation device or sphygmomanometer.

In some examples, the one or more measurement devices 108 may be located at a clinician office or at another location that performs patient measurements, such as a pharmacy or a blood donation center. In another example, the one or more measurement devices 108 may be included within an in-home patient testing device. The one or more measurement devices 108 may be in communication with a computing device, such as a desktop computer of a clinician office or a smart phone of the patient 104.

The cardiovascular risk assessment system 120 may include one or more processors 122 and memory 160 coupled to the one or more processors 122. One or both of the one or more processors 122 or the memory 160 may be coupled to the one or more measurement devices 108. In some examples, the cardiovascular risk assessment system 120 may be co-located with the one or more measurement devices 108. For example, the one or more measurement devices 108 and the cardiovascular risk assessment system 120 may be co-located at a clinician office or at another location that performs patient measurements, such as a pharmacy or a blood donation center. In another example, the one or more measurement devices 108 and the cardiovascular risk assessment system 120 may be integrated within an in-home patient testing device. In some such examples, the one or more measurement devices 108 may be coupled to the cardiovascular risk assessment system 120 via one or more of a wired connection, a wireless local area network (WLAN), or a wireless personal area network (WPAN), as illustrative examples. In some other examples, the cardiovascular risk assessment system 120 may be implemented using a cloud computing architecture, such as where the cardiovascular risk assessment system 120 includes or corresponds to a cloud server. In some such examples, the cardiovascular risk assessment system 120 may be coupled to the one or more measurement devices 108 via a wireless wide area network (WWAN), such as a cellular network or a satellite-based network, as illustrative examples.

The one or more processors 122 may include or may execute one or more of a pre-processing stage 124, a feature selector 128, a feature mask 132, or an artificial intelligence engine 136. Further, the AI engine 136 may include an input handling stage 140 and a multi-head attention module 144.

In some implementations, the AI engine 136 may include or may be referred to as a convolutional neural network (CNN)-free transformer, a one-dimensional (1D) transformer, a 1D CNN-free transformer, a former, or a multi-sequence former. To illustrate, in some implementations, the AI engine 136 may include or may correspond to a recurrent neural network (RNN), such as a gated recurrent unit (GRU) model or a long short-term memory (LSTM) model. In another example, the AI engine 136 may include or may correspond to a transformer-based neural network, such as an audio spectrogram transformer (AST) model. In another example, the AI engine 136 may include or may correspond to feedforward neural network, such as a multilayer perceptron (MLP) model. Other examples are also within the scope of the disclosure.

During operation, the one or more measurement devices 108 may generate patient measurement data 116 associated with one or more patients, such as a patient 104. The patient measurement data 116 may include or may indicate data associated with the cardiovascular system of the patient 104. The patient measurement data 116 may indicate Korotkoff sounds (K-sounds) 118 of the patient 104. To illustrate, the patient measurement data 116 may indicate characteristics of the K-sounds 118 for one or more heartbeats of the patient 104. Each such heartbeat may be associated with one or more different types of K-sounds of the K-sounds 118. The different types may include, for example, K-sound phases I, II, III, IV, and V.

In some examples, the one or more measurement devices 108 may include an auscultation device, such as a stethoscope, used to generate the patient measurement data 116. Alternatively, or in addition, the one or more measurement devices 108 may include a sphygmomanometer, such as an arm-worn blood pressure gauge, used to generate the patient measurement data 116. Further, the one or more measurement devices 108 may include an audio recording device, such as a microphone. For example, an output of the auscultation device or sphygmomanometer may be connected to an input of the audio recording device such that the audio recording device is able to record audio detected by the auscultation device or sphygmomanometer. The patient measurement data 116 may include or may indicate such recorded audio data.

Depending on the example, the patient measurement data 116 may be measured during a single measurement period or during multiple measurement periods. For example, the single measurement period may occur during a single physical examination of the patient 104, during a daily in-home blood pressure reading of the patient 104, or during a daily in-home heart rate reading of the patient 104, as illustrative examples. In some other examples, the patient measurement data 116 may include measurements associated with multiple measurement periods, such as multiple annual physical examinations of the patient 104, multiple daily in-home blood pressure readings of the patient 104, or multiple daily in-home heart rate readings of the patient 104, as illustrative examples.

The cardiovascular risk assessment system 120 may receive at least a portion of the patient measurement data 116. In some examples, the cardiovascular risk assessment system 120 may receive the patient measurement data via a wired connection with the one or more measurement devices 108 or via a wireless connection with the one or more measurement devices 108, such as a WLAN, a WPAN, or a WWAN. In another example, the cardiovascular risk assessment system 120 may receive the patient measurement data 116 via a server, such as a cloud storage server. For example, the one or more measurement devices 108 may upload the patient measurement data 116 to the cloud storage server, and the cardiovascular risk assessment system 120 may retrieve the patient measurement data 116 from the cloud storage server. In some implementations, the one or more measurement devices 108 may alert the cardiovascular risk assessment system 120 upon uploading of the patient measurement data 116 to the cloud storage server to enable the cardiovascular risk assessment system 120 to retrieve the patient measurement data 116.

Further, in some examples, the one or more measurement devices 108 may encrypt the patient measurement data 116 (e.g., to comply with any applicable regulations regarding patient privacy). In one example, the patient measurement data 116 may be encrypted using a public key encryption scheme. In some such examples, the cardiovascular risk assessment system 120 may receive a public key associated with the one or more measurement devices 108. The one or more measurement devices 108 may encrypt the patient measurement data 116 using a private key corresponding to the public key. The one or more measurement devices 108 may receive the encrypted version of the patient measurement data 116 and may decrypt the encrypted version of the patient measurement data 116 using the public key to generate a decrypted version of the patient measurement data 116.

The cardiovascular risk assessment system 120 may generate, based on the patient measurement data 116, an output 150 indicating a patient cardiovascular risk assessment 154 that is based on the K-sounds 118. For example, the cardiovascular risk assessment system 120 may detect one or more abnormalities (or a lack of such abnormalities) associated with the K-sounds 118. In some such examples, the patient cardiovascular risk assessment 154 may have one of a first value (such as a logic one value) indicating that one or more such abnormalities are detected or a second value (such as a logic zero value) indicating that no such abnormalities are detected. Other examples are also within the scope of the disclosure. For example, in another implementation, the patient cardiovascular risk assessment 154 may include a vector or a percentage metric that quantifies an amount of risk associated with the K-sounds 118. For example, an a degree of abnormalities associated with the K-sounds 118 increases, the magnitude of the vector or percentage metric may increase.

In some examples, the output 150 may include or may trigger an alert or a notification. The alert or notification may be presented to the patient 104. To illustrate, in one example, the cardiovascular risk assessment system 120 may correspond to an in-home patient testing device that includes a user interface, such as a graphical user interface (GUI), an auditory user interface, or both. In one example, the user interface may generate an alert by triggering a red light emitting diode (LED) to indicate that the patient cardiovascular risk assessment 154 indicates that one or more such abnormalities are detected or by triggering a green LED to indicate that the patient cardiovascular risk assessment 154 indicates that no such abnormalities are detected. Alternatively, or in addition, the output 150 may include a notification by pushing the notification to a computing device, such as a server or a smartphone of the patient 104. In some such examples, the notification may correspond to an email, a text message, or a push notification associated with an application (app) executed by the smartphone of the patient 104.

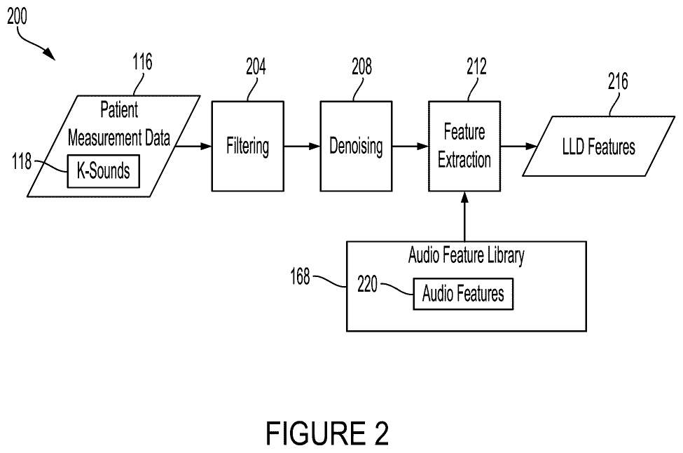

In some examples, the one or more processors 122 may retrieve instructions 164 from the memory 160 and may execute the instructions 164 to initiate, perform, or control one or more operations described herein. Further, in some examples, the memory 160 (or another memory, such as a memory of a cloud server) may store an audio feature library 168. The one or more processors 122 may access the audio feature library 168 to identify features associated with the K-sounds 118, such as described further with reference to the example of FIG. 2.

FIG. 2 shows example pre-processing operations 200 in accordance with some aspects of the disclosure. In some examples, the pre-processing operations 200 of FIG. 2 may be performed by the pre-processing stage 124 of FIG. 1. For example, the pre-processing stage 124 may perform pre-processing of the patient measurement data 116 using one or more pre-processing operations of FIG. 2, such as by performing one or more of filtering 204 of the patient measurement data 116, denoising 208 of the patient measurement data 116, or feature extraction 212 of the patient measurement data 116.

To illustrate, in some examples, the filtering 204 may include applying the patient measurement data 116 to a filter having a frequency response that corresponds to or that is based on a frequency range associated with the K-sounds 118. In some examples, the filter may correspond to a finite impulse response (FIR) filter, such as an FIR bandpass filter or an FIR low-pass filter. To further illustrate, the filter may correspond to a low-pass filter that passes frequences from 0 hertz (Hz) to 100 Hz, 200 Hz, or 300 Hz. In another example, the filter may correspond to a bandpass filter that passes frequencies between 20 Hz and 100, 200, or 300 Hz. Other examples are also within the scope of the disclosure.

In some examples, performing the denoising 208 of the patient measurement data 116 may remove or reduce noise content from the patient measurement data 116. To illustrate, in some implementations, a particular type of measurement modality used to generate the patient measurement data 116 may be associated with a particular type of noise (e.g., a noise signature). For example, if the one or more measurement devices 108 are powered by a mains supply (such as in the case of a mains-supplied sphygmomanometer), the patient measurement data 116 may be associated with 60 Hz noise. As another example, in some implementations, breathing by the patient 104 may introduce breathing artifacts in the patient measurement data 116. Accordingly, the denoising 208 may include evaluating the patient measurement data 116 for such noise and removing or attenuating the noise from the patient measurement data 116.

In some examples, performing the feature extraction 212 may include accessing the audio feature library 168. The audio feature library 168 may indicate a plurality of audio features 220 associated with K-sounds, such as an audio “signature” of K-sounds. In some implementations, the audio feature library 168 may be determined by measuring K-sounds produced by a large quantity of individuals and identifying common features of the K-sounds in the individuals. To further illustrate, the audio feature library 168 may indicate properties such as one or more of pitch, loudness, Mel-Frequency Cepstral coefficients (MFCCs), or spectral properties associated with K-sounds.

Performing the feature extraction 212 may further include parsing the patient measurement data 116 to identify at least a subset of audio features of the plurality of audio features 220. Performing the feature extraction 212 may also include extracting the subset of audio features from the patient measurement data 116 to generate low-level descriptor (LLD) features 216. The LLD features 216 may include, for example, one or more of spectral features associated with the K-sounds 118, cepstral features associated with the K-sounds 118, energy features associated with the K-sounds 118, frequency domain associated with the K-sounds 118, or time domain features associated with the K-sounds 118.

In some examples, the LLD features 216 may include or may be referred to as time series data. To illustrate, performing the feature extraction 212 may include applying a window (e.g., a sliding or moving window) to the patient measurement data 116 to search for the audio features 220 within the patient measurement data 116 (e.g., where the window defines a search area within the patient measurement data 116). In some cases, the feature extraction 212 may use multiple different time series characteristics. For example, the multiple different time series characteristics may include window size (e.g., a duration of a window), step size (e.g., an amount the window moves, slides, or “steps”), or both. In some examples, using such different time series characteristics may improve performance associated with the feature extraction 212, such as by increasing a quantity of LLD features extracted from the patient measurement data 116, increasing accuracy of the feature extraction 212, or both.

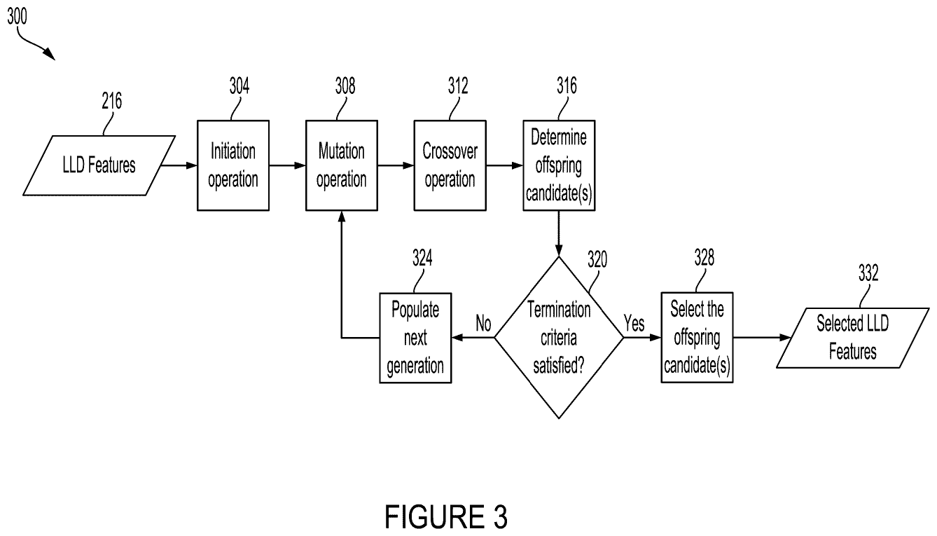

FIG. 3 shows an example of a genetic selection procedure 300 in accordance with some aspects of the disclosure. In some examples, one or more operations of the genetic selection procedure 300 may be performed by the feature selector 128 of FIG. 1. Further, the genetic selection procedure 300 may be performed based on a first plurality of LLD features, such as the LLD features 216 of FIG. 2. In some examples, the genetic selection procedure 300 may include or may be referred to as a differential evolution (DE) procedure.

The genetic selection procedure 300 may include performing a population initiation operation 304. In some examples, performing the population initiation operation 304 may include introducing the LLD features 216 as candidates of the genetic selection procedure 300, such as by inputting the LLD features 216 to the feature selector 128 of FIG. 1. The LLD features 216 may also be referred to as “genes” associated with the genetic selection procedure 300. Further, in some examples, a “candidate” may refer to a subset of features of the LLD features 216, such as a randomly selected subset of the LLD features 216, and a “population” may refer to a group of candidates. Accordingly, in some examples, performing the population initiation operation 304 may include determining a group of different respective randomly selected subsets of the LLD features 216.

The genetic selection procedure 300 may further include a mutation operation 308. In some examples, performing the mutation operation 308 may include introducing mutated candidates to the genetic selection procedure 300. For example, the feature selector 128 of FIG. 1 may modify one or more characteristics of at least one of the LLD features 216. In some examples, the one or more characteristics may include an amplitude of an LLD feature, a frequency characteristic of an LLD feature, a phase characteristic of an LLD feature, or another characteristic. Performing the mutation operation 308 may introduce diversity to the LLD features 216. For example, the LLD features 216 may be referred to as candidates (or “genes”) associated with the genetic selection procedure 300, and performing the mutation operation 308 may include modifying the genes to generate mutated candidates (or mutated candidate genes).

The genetic selection procedure 300 may further include a crossover operation 312. In some examples, performing the crossover operation 312 may include combining at least some of the candidates with at least some of the mutated candidates to generate offspring candidates. For example, the offspring candidates may correspond to a new “generation” of candidates that inherit genes from both the candidates with the mutated candidates.

The genetic selection procedure 300 may further include performing a selection operation 316, such as by selecting one or more of the offspring candidates based on one or more performance criteria. For example, the offspring candidates may be ranked according to classification accuracy, and one or more higher-ranking candidates may be selected from among the offspring candidates. In such examples, the one or more performance criteria may include a ranking of classification accuracy.

The genetic selection procedure 300 may further include determining whether one or more termination criteria are satisfied, at 320. For example, the one or more termination criteria may include a threshold quantity of iterations associated with the genetic selection procedure 300, one or more threshold performance criteria to be achieved in connection with a generation of candidate offspring, one or more other termination criteria, or a combination thereof. In some examples, the feature selector 128 may detect that that the one or more termination criteria are not satisfied (e.g., based on a determination that a current quantity of iterations associated with the genetic selection procedure 300 fails to exceed the threshold quantity). Based on detecting that the one or more termination criteria are not satisfied, the genetic selection procedure 300 may continue, such as by performing an additional iteration of the mutation operation 308. During the additional iteration of the mutation operation 308, an additional generation of offspring candidates may inherit genes from the current generation of offspring candidates.

In some other examples, the feature selector 128 may detect that that the one or more termination criteria are satisfied (e.g., based on a determination that a current quantity of iterations associated with the genetic selection procedure 300 exceeds the threshold quantity). In such examples, the genetic selection procedure 300 may further include selecting, based on detecting that one or more termination criteria are satisfied, the one or more offspring candidates as a second plurality of LLD features, at 328. The second plurality of LLD features may correspond to selected LLD features 332.

In some examples, one or more operations of the genetic selection procedure 300 may be performed in connection with feature relevance scores, which may be determined using an ensemble learning procedure. An ensemble learning procedure may use multiple models. For example, a “boosted” ensemble learning procedure may use multiple models cooperatively (e.g., where a subsequent model may adjust or correct an output of a prior model). An example of a boosted ensemble learning procedure is an adaptive boosting (AdaBoost) classifier. To further illustrate, in some examples, the genetic selection procedure 300 may be performed in accordance the following example operations:

### Initialization Operation 304

Generate candidates randomly with different initial feature subsets to form a population (P) with the size (P_size).
Pass those candidates to an AdaBoost classifier to determine their feature relevance scores.

### Mutation Operation 308 and Crossover Operation 312

Generate offspring candidates by randomly picking two parents from the population (P) and applying crossover on their feature subsets.
Construct a mutant vector randomly, based on a weighted threshold.
Modify the offspring candidates using the mutant vector to create a new population (P′) with the size (P_size′).
Evaluate those offspring candidates using the AdaBoost classifier to determine their feature relevance scores.

### Selection Operation 316

Merge the two populations (P+P′).
Sort the combined population based on the feature relevance scores in ascending order.
Select the top candidates within P_size to form the next population (P″).
Repeat the “Crossover+Mutation” step until one of the termination criteria is met.

### Termination Criteria 320:

The iteration number meets a threshold iteration number (such as 1000 or another number); or
The average feature relevance score of population P″ is within a threshold amount of the highest candidate score of population P″.

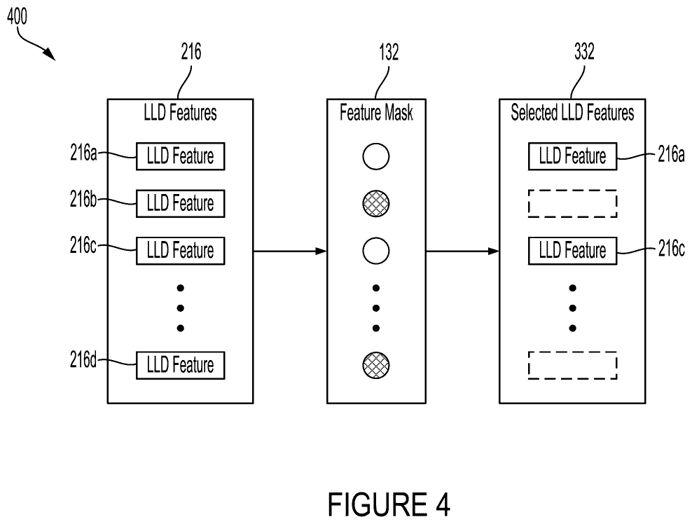

In some examples, the candidate with the highest feature relevance scores may be used to generate an LLD feature mask, such as the feature mask 132 of FIG. 1. The feature mask 132 may be used to generate the selected LLD features 332. Some examples that may be associated with the feature mask 132 are described further with reference to FIG. 4.

FIG. 4 shows an example of a masking operation 400 in accordance with some aspects of the disclosure. The masking operation 400 may be performed using a feature mask, such as the feature mask 132 of FIG. 1. In some examples, the masking operation 400 may be used to perform the selection operation, at 328, of FIG. 3.

The LLD features 216 may be provided to the feature mask 132. In the example of FIG. 4, the LLD features 216 may include LLD features 216a, 216b, 216c, and 216n, where “n” may correspond to a positive integer that indicates a quantity of LLD features included in the LLD features 216. Further, the feature mask 132 may correspond to a candidate of the genetic selection procedure 300 of FIG. 3 having the highest feature relevance scores. To illustrate, the candidate may include the LLD features 216a and 216c and may exclude the LLD features 216b and 216n. Accordingly, the feature mask 132 may include a logic one bit in position one, a logic zero bit in position two, a logic one bit in position three, and a logic zero bit in position n.

The feature mask 132 may be applied to the LLD features 216 to determine a subset of the LLD features 216, such as the selected LLD features 332. In some examples, applying the feature mask 132 to the LLD features 216 may include masking (e.g., excluding) at least one LLD feature of the LLD features 216. To illustrate, in the example of FIG. 4, applying the feature mask 132 to the LLD features 216 may include masking (e.g., excluding) the LLD feature 216b and the LLD feature 216n. In such examples, the selected LLD features 332 may include the LLD feature 216a and the LLD feature 216c and may exclude the LLD feature 216b and the LLD feature 216n. Other examples are also within the scope of the disclosure.

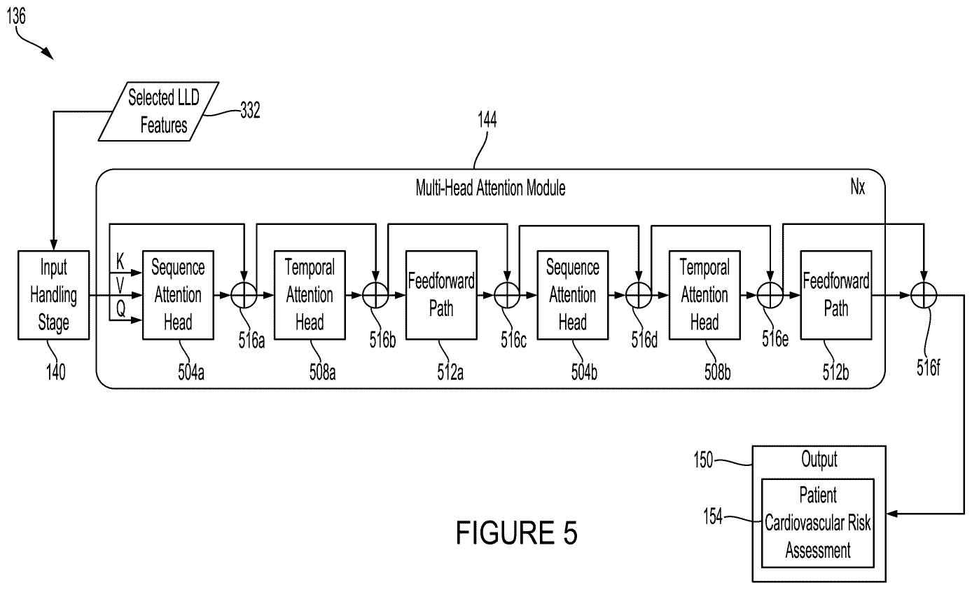

FIG. 5 shows examples of features that may be associated with the AI engine 136 in accordance with some aspects of the disclosure. The AI engine 136 may include the input handling stage 140 and the multi-head attention module 144. An output of the input handling stage 140 may be coupled to an input of the multi-head attention module 144.

In the example of FIG. 5, the multi-head attention module 144 may include multiple sequence attention heads, multiple temporal attention heads, and multiple paths coupling the multiple sequence attention heads to the multiple temporal attention heads. For example, the multi-head attention module 144 may include sequence attention heads 504a-b and temporal attention heads 508a-b. The sequence attention heads 504a-b may be referred to as a multi-head sequence attention module, and the temporal attention heads 508a-b may be referred to as a multi-head temporal attention module. The multi-head attention module 144 may also include summation nodes 516a-e and feedforward paths 512a-b. The feedforward paths 412a-b may include or may be referred to as feedforward layers. An output of the multi-head attention module 144 may be coupled to summation node 516f.

The sequence attention head 504a may be coupled to the summation node 516a. For example, an input of the sequence attention head 504a and an output of the sequence attention head 504a may be coupled to inputs of the summation node 516a. An output of the summation node 516a may be coupled to an input of the temporal attention head 508a. The input of the temporal attention head 508a and an output of the temporal attention head 508a may be coupled to inputs of the summation node 516b. An output of the summation node 516b may be coupled to an input of the feedforward path 512a.

The input of the feedforward path 512a and an output of the feedforward path 512a may be coupled to inputs of the summation node 516c. An output of the summation node 516c may be coupled to an input of the sequence attention head 504b. The input of the sequence attention head 504b and an output of the sequence attention head 504b may be coupled to inputs of the summation node 516d. An output of the summation node 516d may be coupled to an input of the temporal attention head 508b. The input of the temporal attention head 508b and an output of the temporal attention head 508b may be coupled inputs of to the summation node 516e. An output of the summation node 516e may be coupled to an input of the feedforward path 512b. Inputs of the summation node 516f may be coupled to an output of the feedforward path 512b and to the output of the summation node 516e.

Although the example of FIG. 5 illustrates that the multi-head attention module may include two sequence attention heads, two temporal attention heads, and two feedforward paths, other examples are also within the scope of the disclosure. To illustrate, in some examples, the multi-head attention module 144 may include N copies of, or N times (N×), the architecture illustrated in FIG. 5, where N is a positive integer. In some examples, the N copies may be coupled in parallel with one another or in series with one another.

During operation, the input handling stage 140 may perform input handling associated with LLD features, such as the selected LLD features 332. In some examples, the input handling stage 140 may tokenize the selected LLD features 332 to generate tokenized LLD features and may perform embedding associated with the tokenized LLD features. The input handling stage 140 may provide the tokenized LLD features to the multi-head attention module 144, such as by providing the tokenized LLD features as query (Q), key (K), and value (V) matrices in accordance with a query-key-value (QKV) format.

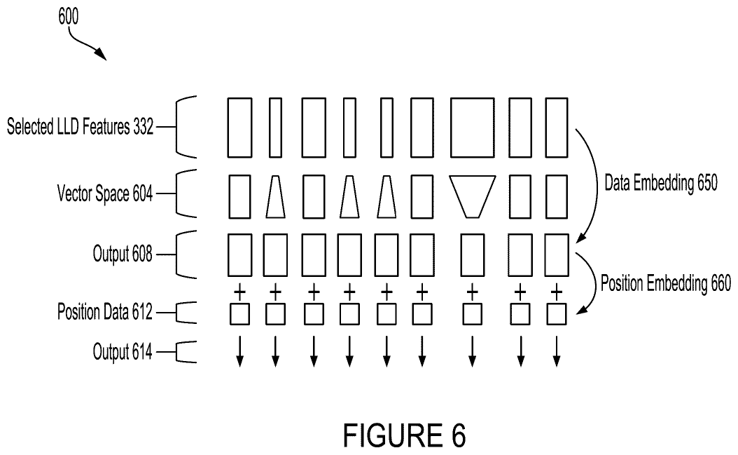

In some examples, the input handling stage 140 may handle LLD features associated with multiple different time series. For example, the selected LLD features 332 may include multiple time series of LLD features. Further, the input handling stage 140 may process multiple time series inputs simultaneously, which may enable efficient handling of diverse data streams. In some implementations, the input handling stage 140 may support time series of multiple different time series characteristics, which may include, for example, different window sizes, different step sizes, or both, as described with reference to the feature extraction 212 of FIG. 2. In some examples, the input handling stage 140 may project data of multiple different time series characteristics into a common type or format for unified processing, which may enable processing of a variety of different data types, formats, and complexities. To further illustrate, the input handling stage 140 may embed the selected LLD features 332 into a vector space, such as a 1D vector space, and performing the embedding may include projecting data from different dimensions into a common dimension associated with the vector space. As an illustrative example, the selected LLD features 332 may include at least a first feature associated with a first dimension (such as a first number of rows, a first number of columns, or both) and a second feature associated with a second dimension different than the first dimension (such as a second number of rows different than the first number of rows, a second number of columns different than the first number of columns, or both). The input handling stage 140 may project one or more of the first feature or the second feature into a common dimension, such as a common number of rows, a common number of columns, or both. Some illustrative examples operations that may be associated with the input handling stage 140 are described further with reference to FIG. 6.

The multi-head attention module 144 may receive information from the input handling stage. The multi-head attention module 144 may determine sequence attention scores (e.g., using the sequence attention heads 504a-b) and may determine temporal attention heads (e.g., using the temporal attention heads 508a-b). To illustrate, the multi-head attention module 144 may include multiple sequence attention heads (such as the sequence attention heads 508a-b) that determine the sequence attention scores in parallel based at least in part on one or more of inter-sequence dependencies associated with sequences of features, relationships between the sequences, or interactions between the sequences. The sequence attention scores may indicate an amount of focus given to features relative to one another, where such features are associated with a common time point (or timestamp). For example, after processing of the selected LLD features 332 by the input handling stage 140 (e.g., embedding of the selected LLD features 332 to generate embedded features), the input handling stage 140 may provide the embedded features as K, V, and Q parameters to the multi-head attention module 144, and the multi-head attention module 144 may determine the sequence attention scores based on inter-sequence dependencies associated with sequences of the K, V, and Q parameters, relationships between the sequences, or interactions between the sequences. In some examples, the sequence attention heads 508a-b may determine an amount of focus or weight given to input features for a particular timestamp.

Further, the multi-head attention module 144 may include multiple temporal attention heads (such as the temporal attention heads 508a-b) that operate along a time dimension and that determine the temporal attention scores based at least in part on changes or trends associated with the K-sounds 118 during a common time interval. The temporal attention scores may each indicate how a particular feature relates to past and future values of the feature. In some examples, the temporal attention heads 508a-b may process different respective patterns associated with the K-sounds 118. Accordingly, the multi-head attention module 144 may perform parallel processing of sequences of K, V, and Q parameters associated with a common timestamp (e.g., using the sequence attention heads 504a-b) and may also perform parallel processing of K, V, and Q parameters to determine changes or trends associated with the K-sounds 118 (e.g., using the temporal attention heads 508a-b).

In some examples, the feedforward paths 512a-b may perform transformation operations associated with outputs of attention heads of the multi-head attention module 144. For example, the transformation operations may include a linear layer up projection (e.g., to expand data from a one-dimensional space into a higher-dimensional space), activation (e.g., to add or increase non-linearity), or a linear layer down projection (e.g., to reduce data from the higher-dimensional space to the one-dimensional space), as illustrative examples. Further, values generated by the multi-head attention module 144 may be summed by the summation node 516f to generate the output 150. The output 150 may indicate the patient cardiovascular risk assessment 154. For example, the feedforward path 512b may apply final linear translation layers to map processed features from prior stages of the multi-head attention module 144 to a risk score, such as the patient cardiovascular risk assessment 154.

FIG. 6 shows an example of an embedding process 600 in accordance with some aspects of the disclosure. In some examples, the input handling stage 140 may perform the embedding process 600. Further, the embedding process 600 may include one or both of data embedding 650 or position embedding 660.

During the embedding process 600, the input handling stage 140 may receive the selected LLD features 332 and may embed the selected LLD features 332 into a vector space 604. Performing the data embedding 650 may include simultaneously embedding tokens representing the selected LLD features 332 into the vector space 604 to generate a first output, such as output 608. The data embedding 650 may also be referred to as tokenization or token encoding. Further, performing the position embedding 660 may include simultaneously adding position data 612 to the first output to generate a second output, such as output 614. In some examples, the position data 612 may represent positions associated with the output 608, such as positions of embedded tokens relative to one another in the output 608.

In some examples, the embedding process 600 may further include providing the output 614 to the multi-head attention module 144. For example, the output 614 may include query (Q), key (K), and value (V) matrices, which may be provided to the multi-head attention module 144 in accordance with a query-key-value (QKV) format.

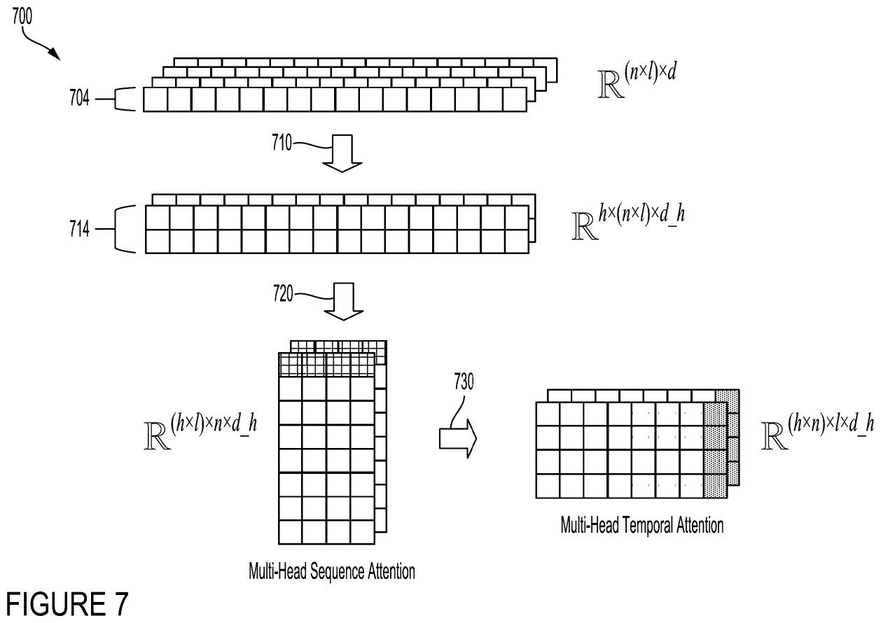

FIG. 7 shows examples of operations 700 in accordance with some aspects of the disclosure. In some examples, the multi-head attention module 144 may perform the operations 700.

The operations 700 may include receiving a set of l layers, where each layer of the set of layers includes n tokens. The n tokens may correspond to tokenized versions of LLD features, such as the selected LLD features 332. Each token may be associated with a model dimension d. In some examples, d=1. In FIG. 7, the l layers may include a representative layer 704, and each square may represent one of the n tokens. In each layer, tokens may be concatenated with one another. Further, the l layers may be represented in accordance with Expression 1:

[symbol]

^{(n×l)×d} (Expression 1).

In Expression 1, [symbol] may indicate the set of real numbers.

The operations 700 may include performing a stacking operation, at 710. The stacking operation may generate a set of stacked layers, such as a representative stacked layer 714. The set of stacked layers may be represented by Expression 2:

[symbol]

^{h×(n×l)×d_h} (Expression 2).

In Expression 2, h may represent h may indicate a quantity of heads (also referred to as attention heads) of the multi-head attention module 144, n×l may indicate a total quantity of tokens across all layers, and d_h may indicate a dimensionality of each head of the multi-head attention module 144.

The operations 700 may further include determining sequence attention scores, at 720. The sequence attention may be represented using Expression 3:

[symbol]

^{(h×l)×n×d_h} (Expression 3).

To further illustrate, determining the sequence attention scores may include performing, by the multi-head attention module 144, attention scoring on the same time point across different sequences of the selected LLD features 332. By analyzing such different sequences, the multi-head attention module 144 may detect relationships between different LLD features, such as inter-sequence dependencies. Such dependencies may improve accuracy of detecting or classifying risk of cardiovascular disease, such as heart failure (HF). Moreover, by using multiple attention heads (such as the sequence attention heads 504), the multi-head attention module 144 may gain a deeper understanding of the interactions between sequences of different LLD features, leading to more accurate and nuanced representation of the patient measurement data 116.

The operations 700 may further include determining temporal attention scores, at 730. The temporal attention may be represented using Expression 4:

[symbol]

^{(h×n)×l×d_h} (Expression 4).

To further illustrate, determining the temporal attention scores may include performing, by the multi-head attention module 144, attention scoring along the time dimension, which may enable the multi-head attention module 144 to focus on temporal dynamics present within each sequence of the selected LLD features 332. By analyzing the progression of features over time, the multi-head attention module 144 may detect and weight changes and trends associated with different time points that may indicate the presence of heart failure. Moreover, by using multiple heads (such as the temporal attention heads 508), the multi-head attention module 144 may concentrate on different temporal patterns, which may facilitate a fine-grained analysis of time series data. Such a fine-grained analysis may increase temporal sensitivity, which may be advantageous in detecting subtle variations in K-sounds that may be associated with risk of cardiovascular disease, such as heart failure.

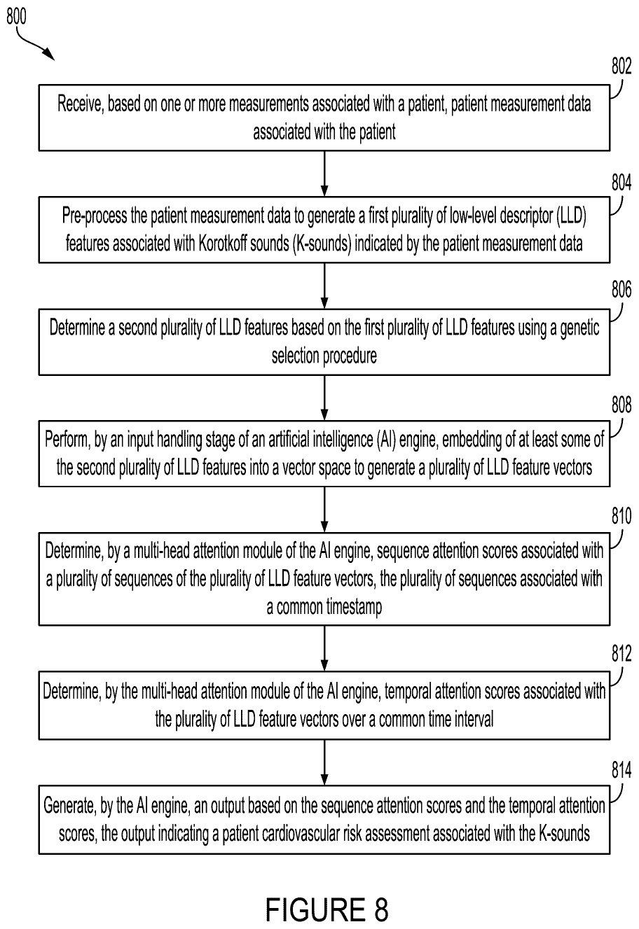

FIG. 8 shows an example of a method 800 in accordance with some aspects of the disclosure. In some examples, one or more operations of the method 800 may be performed by the cardiovascular risk assessment system 120 of FIG. 1. In some examples, a processing system may include one or more processors (e.g., the one or more processors 122) and one or more memories coupled to the one or more processors (e.g., the memory 160) and may be configured to perform one or more operations of the method 800.

The method 800 includes receiving, based on one or more measurements associated with a patient, patient measurement data associated with the patient, at 802. For example, the patient measurement data may correspond to the patient measurement data 116, which may be generated via one or more measurements of the patient 104 using the one or more measurement devices 108.

The method 800 further includes pre-processing the patient measurement data to generate a first plurality of low-level descriptor (LLD) features associated with Korotkoff sounds (K-sounds) indicated by the patient measurement data, at 804. For example, the pre-processing stage 124 may pre-process the patient measurement data 116 to generate the LLD features 216 of FIG. 2 that are associated with the K-sounds 118 indicated by the patient measurement data 116. In this example, the first plurality of LLD features may include or correspond to the LLD features 216.

The method 800 further includes determining a second plurality of LLD features based on the first plurality of LLD features using a genetic selection procedure, at 806. For example, the genetic selection procedure may include or correspond to the genetic selection procedure 300 of FIG. 3, and the second plurality of LLD features may include or correspond to the selected LLD features 332 of FIG. 3.

The method 800 further includes performing, by an input handling stage of an artificial intelligence (AI) engine, embedding of at least some of the second plurality of LLD features into a vector space to generate a plurality of LLD feature vectors, at 808. For example, the embedding may include or may correspond to the embedding process 600 of FIG. 6, and the plurality of LLD feature vectors may include or may correspond to the output 614 of FIG. 6.

The method 800 further includes determining, by a multi-head attention module of the AI engine, sequence attention scores associated with a plurality of sequences of the plurality of LLD feature vectors, the plurality of sequences associated with a common timestamp, at 810. For example, the multi-head attention module 144 may receive the plurality of LLD feature vectors from the input handling stage 140 and may determine the sequence attention scores using the sequence attention heads 504a-b based on sequences of the plurality of LLD feature vectors for a particular timestamp. In some examples, the sequence attention scores may correspond to the sequence attention scores described with reference to FIG. 7, such as in connection with Expression 3.

The method 800 further includes determining, by the multi-head attention module of the AI engine, temporal attention scores associated with the plurality of LLD feature vectors over a common time interval, at 812. For example, the multi-head attention module 144 may receive the plurality of LLD feature vectors from the input handling stage 140 and may determine the temporal attention scores using the temporal attention heads 508a-b based on sequences of the plurality of LLD feature vectors over a particular time interval. In some examples, the temporal attention scores may correspond to the temporal attention scores described with reference to FIG. 7, such as in connection with Expression 4.

The method 800 further includes generating, by the AI engine, an output based on the sequence attention scores and the temporal attention scores, the output indicating a patient cardiovascular risk assessment associated with the K-sounds, at 814. For example, the output may correspond to the output 150 and may indicate the patient cardiovascular risk assessment 154.

Although the present invention and its advantages have been described in detail, it should be understood that various changes, substitutions, and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

Moreover, the scope of the present application is not intended to be limited to the particular embodiments of the process, machine, manufacture, composition of matter, means, methods and steps described in the specification.

What is claimed is:

1. A method comprising:
receiving, based on one or more measurements associated with a patient, patient measurement data associated with the patient;
pre-processing the patient measurement data to generate a first plurality of low-level descriptor (LLD) features associated with Korotkoff sounds (K-sounds) indicated by the patient measurement data;
determining a second plurality of LLD features based on the first plurality of LLD features using a genetic selection procedure;
performing, by an input handling stage of an artificial intelligence (AI) engine, embedding of at least some of the second plurality of LLD features into a vector space to generate a plurality of LLD feature vectors;
determining, by a multi-head attention module of the AI engine, sequence attention scores associated with a plurality of sequences of the plurality of LLD feature vectors, the plurality of sequences associated with a common timestamp;
determining, by the multi-head attention module of the AI engine, temporal attention scores associated with the plurality of LLD feature vectors over a common time interval; and
generating, by the AI engine, an output based on the sequence attention scores and the temporal attention scores, the output indicating a patient cardiovascular risk assessment associated with the K-sounds.

2. The method of claim 1, wherein pre-processing the patient measurement data includes one or more of:
filtering the patient measurement data using a filter having a frequency response that corresponds to or that is based on a frequency range associated with the K-sounds;
denoising the patient measurement data to remove or reduce noise content from the patient measurement data; or
performing feature extraction associated with the patient measurement data.

3. The method of claim 2, wherein performing the feature extraction includes:
accessing an audio feature library that indicates a plurality of audio features associated with the K-sounds;
parsing the patient measurement data to identify at least a subset of audio features of the plurality of audio features; and
extracting the subset of audio features from the patient measurement data to generate the first plurality of LLD features.

4. The method of claim 1, wherein determining the second plurality of LLD features using the genetic selection procedure includes performing one or more of:
a population initiation operation that includes introducing the first plurality of LLD features as candidates of the genetic selection procedure;
a mutation operation that includes introducing mutated candidates to the genetic selection procedure;
a crossover operation that includes combining at least some of the candidates with at least some of the mutated candidates to generate offspring candidates;
selecting one or more of the offspring candidates based on one or more performance criteria; or
based on detecting that one or more termination criteria are satisfied, selecting the one or more offspring candidates as the second plurality of LLD features.

5. The method of claim 1, wherein the vector space is a one-dimensional (1D) vector space, and wherein embedding at least some of the second plurality of LLD features into the vector space includes projecting data from different dimensions into a common dimension associated with the vector space.

6. The method of claim 1, wherein embedding the second plurality of LLD features into the vector space includes performing one or more of data embedding associated with the second plurality of LLD features or position embedding associated with the second plurality of LLD features.

7. The method of claim 6, wherein performing the data embedding includes simultaneously embedding tokens representing the second plurality of LLD features into the vector space to generate a first output, and wherein performing the position embedding includes simultaneously adding position data to the first output to generate a second output corresponding to the plurality of LLD feature vectors.

8. The method of claim 1, wherein the multi-head attention module includes multiple sequence attention heads that determine the sequence attention scores in parallel based at least in part on one or more of inter-sequence dependencies associated with the sequences, relationships between the sequences, or interactions between the sequences.

9. The method of claim 1, wherein the multi-head attention module includes multiple temporal attention heads that determine the temporal attention scores based at least in part on changes or trends associated with the K-sounds during the common time interval, and wherein the multiple temporal attention heads process different respective patterns associated with the K-sounds.

10. The method of claim 1, further comprising:
providing the first plurality of LLD features to a feature mask; and
applying the feature mask to the first plurality of LLD features to determine a subset of the first plurality of LLD features, wherein the subset corresponds to the second plurality of LLD features.

11. An apparatus comprising:
a processing system including one or more processors and one or more memories coupled to the one or more processors, the processing system configured to:
receive, based on one or more measurements associated with a patient, patient measurement data associated with the patient;
pre-process the patient measurement data to generate a first plurality of low-level descriptor (LLD) features associated with Korotkoff sounds (K-sounds) indicated by the patient measurement data;
determine a second plurality of LLD features based on the first plurality of LLD features using a genetic selection procedure;
perform, by an input handling stage of an artificial intelligence (AI) engine, embedding of at least some of the second plurality of LLD features into a vector space to generate a plurality of LLD feature vectors;
determine, by a multi-head attention module of the AI engine, sequence attention scores associated with a plurality of sequences of the plurality of LLD feature vectors, the plurality of sequences associated with a common timestamp;
determine, by the multi-head attention module of the AI engine, temporal attention scores associated with the plurality of LLD feature vectors over a common time interval; and
generate, by the AI engine, an output based on the sequence attention scores and the temporal attention scores, the output indicating a patient cardiovascular risk assessment associated with the K-sounds.

12. The apparatus of claim 11, wherein pre-processing the patient measurement data includes one or more of:
filtering the patient measurement data using a filter having a frequency response that corresponds to or that is based on a frequency range associated with the K-sounds;
denoising the patient measurement data to remove or reduce noise content from the patient measurement data; or
performing feature extraction associated with the patient measurement data.

13. The apparatus of claim 12, wherein performing the feature extraction includes:
accessing an audio feature library that indicates a plurality of audio features associated with the K-sounds;
parsing the patient measurement data to identify at least a subset of audio features of the plurality of audio features; and
extracting the subset of audio features from the patient measurement data to generate the first plurality of LLD features.

14. The apparatus of claim 11, wherein determining the second plurality of LLD features using the genetic selection procedure includes performing one or more of:
a population initiation operation that includes introducing the first plurality of LLD features as candidates of the genetic selection procedure;
a mutation operation that includes introducing mutated candidates to the genetic selection procedure;
a crossover operation that includes combining at least some of the candidates with at least some of the mutated candidates to generate offspring candidates;
selecting one or more of the offspring candidates based on one or more performance criteria; or
based on detecting that one or more termination criteria are satisfied, selecting the one or more offspring candidates as the second plurality of LLD features.

15. The apparatus of claim 11, wherein the vector space is a one-dimensional (1D) vector space, and wherein embedding at least some of the second plurality of LLD features into the vector space includes projecting data from different dimensions into a common dimension associated with the vector space.

16. The apparatus of claim 11, wherein embedding the second plurality of LLD features into the vector space includes performing one or more of data embedding associated with the second plurality of LLD features or position embedding associated with the second plurality of LLD features.

17. The apparatus of claim 16, wherein performing the data embedding includes simultaneously embedding tokens representing the second plurality of LLD features into the vector space to generate a first output, and wherein performing the position embedding includes simultaneously adding position data to the first output to generate a second output corresponding to the plurality of LLD feature vectors.

18. The apparatus of claim 11, wherein the multi-head attention module includes multiple sequence attention heads that determine the sequence attention scores in parallel based at least in part on one or more of inter-sequence dependencies associated with the sequences, relationships between the sequences, or interactions between the sequences.

19. The apparatus of claim 11, wherein the multi-head attention module includes multiple temporal attention heads that determine the temporal attention scores based at least in part on changes or trends associated with the K-sounds during the common time interval, and wherein the multiple temporal attention heads process different respective patterns associated with the K-sounds.

20. A method of operation of a multi-sequence former for enhanced sequence processing and classification, the method comprising:
receiving, based on one or more measurements associated with a patient, patient measurement data associated with the patient;
pre-processing the patient measurement data to generate a first plurality of low-level descriptor (LLD) features associated with Korotkoff sounds (K-sounds) indicated by the patient measurement data;
determining a second plurality of LLD features based on the first plurality of LLD features using a genetic selection procedure;
performing, by an input handling stage of the multi-sequence former, embedding of at least some of the second plurality of LLD features into a vector space to generate a plurality of LLD feature vectors;
determining, by a multi-head sequence attention module of the multi-sequence former, sequence attention scores associated with a plurality of sequences of the plurality of LLD feature vectors, the plurality of sequences associated with a common timestamp;
determining, by a multi-head temporal attention module of the multi-sequence former, temporal attention scores associated with the plurality of LLD feature vectors over a common time interval; and
generating, by the multi-sequence former, an output based on the sequence attention scores and the temporal attention scores, the output indicating a patient cardiovascular risk assessment associated with the K-sounds.
